(19) United States
(12) Patent Application Publication
(10) Pub. No.: US 2001/0018077 A1
(43) Pub. Date: Aug. 30, 2001

(54) Herbal formulation for stimulating the immune system to prevent colds and the flu and method of using same

(76) Inventor: Edward O. Shaner, Ocean City, MD (US)

Correspondence Address: LOWE HAUPTMAN GILMAN & BERNER, LLP, 1700 Diagonal Road, Suite 310, Alexandria, VA, 22314, US

(21) Appl. No.: 09/739,212

(22) Filed: Dec. 19, 2000

Related U.S. Application Data
(63) Continuation-in-part of application No. 09/488,590, filed on Jan. 24, 2000.

(51) Int. Cl. A61K 35/78; A01N 65/00

(52) U.S. Cl. 424/726; 424/737; 424/769; 424/777; 514/494

(57) ABSTRACT

A herbal formulation that is orally administered and which affects colds or the flu in a human subject comprises beta glucans, olive leaf extract, echinacea, goldenseal, una de gato, pao d'arco, elderberry dried berries and cayenne pepper. The formulation can also include zinc lozenges if symptoms of a sore throat are present in the subject.

### RELATIONSHIP TO PENDING APPLICATION

The present application is a continuation-in-part of applicant's pending U.S. patent application Ser. No. 09/488,590 filed Jan. 24, 2000.

### FIELD OF INVENTION

The present invention relates generally to a method of and/or formula for reducing the effects of colds and the flu and, more particularly, to a combination of herbs for reducing the effects of colds and the flu.

### BACKGROUND OF THE INVENTION

The most powerful and versatile biological defense system in humans is the immune system. It is the system humans depend upon to protect against winter colds and the flu. Cold and flu viruses are predators that enter the cells that line the nose and throat, proliferate, and inflame the host cell. The immune defense system consists of specialized cells called lymphocytes which have the unique ability to recognize, evaluate, and destroy foreign invaders. With advancing age, the immune system declines in effectiveness.

Lymphocytes fall into two classes. The T-lymphocytes which are produced and activated by the thymus gland, and the B-lymphocytes which are produced in bone marrow. T-cells are the watchdogs of the immune system. T-cells are programmed to recognize antigens, which may be foreign cells, bacteria, viruses, large molecules, or combinations. When the T-cells recognize an antigen, the T-cells destroy the invaders directly, or by release of substances called lymphokines. The lymphokines cause other white blood cells to become “angry,” thus increasing their activity to engulf and destroy bacteria and foreign cells.

There are hundreds of common colds and flu viruses. No vaccine exists that provides a common defense. Persons with weakened immune systems are highly susceptible. Herbs with known viricidal properties are echinacea by itself and in combination with goldenseal, una de gato (also known as Cat's Claw) by itself, pao d'arco by itself, elderberry dried berries by themselves, olive leaf extract by itself, beta glucans by themselves, cayenne pepper by itself, vitamin C, garlic perles and beta carotene. In addition, zinc lozenges (zinc gluconate) are a known sore throat suppression agent. Single herbs that exhibit viricidal properties are relatively ineffective against viruses which cause colds and the flu.

### SUMMARY OF THE INVENTION

The herbs in the formula of the present invention contain polysaccharides, bioflavenoids, proanthro-cyanadines, glycosides, and other phytochemicals. In a first preferred embodiment, an herbal formula in accordance with the invention includes beta glucans in combination with at least one of: olive leaf extract, echinacea with and without goldenseal, una de gato, pao d'arco, elderberry dried berries and cayenne pepper. A second preferred embodiment includes olive leaf extract in combination with at least one of beta glucans, echinacea with and without goldenseal, una de gato, pao d'arco, elderberry dried berries and cayenne pepper. A third preferred embodiment of the invention relates to the combination of beta glucans, olive leaf extract, echinacea with and without goldenseal, una de gato, pao d'arco, elderberry dried berries and cayenne pepper. Zinc gluconate, preferably in the form of lozenges, can be added to any of these formulae to assist in sore throat treatment.

When combined together in certain ratios, the herbs in the formulae of the present invention have viricidal components. The viricidal components of all three embodiments combine to prevent or abate incipient colds and the flu. The viricidal components of the third embodiment, when that embodiment is used as directed, work synergistically to potentiate their viricidal effect on colds and flu viruses. The herbs of the third embodiment act synergistically to destroy a wide variety of viruses through an energized immune system.

The colds and flu viruses response to the combination of phytochemicals in the specific listed herbs of all three embodiments is to release chemicals to protect themselves. Lymphokines of a subject ingesting the combination of phytochemicals recognize them as powerful antigens and are stimulated to produce a burst of antibodies.

Colds and flu viruses exhibit a wide variety of chemical barriers to the antibodies produced by the lymphokines. The present invention results from my realization of the necessity to bombard the virus with multiple phytochemicals to overcome the virus defense system. The combinations of the phytochemicals found in the formulae of herbs of all three embodiments provide a wide spread of pathogenicity to colds and flu viruses. The combination of phytochemicals of the third embodiment acts synergistically to potentiate the viricidal action of the individual phytochemical.

### DESCRIPTION OF THE PREFERRED EMBODIMENT OF THE INVENTION

Basic daily prevention:

| | | |
|---|---|---|
| 500 | mg | Vitamin C |
| 1,000 | mg | Garlic perles |
| 15,000 | units | Beta carotene |

Formula of the third embodiment for anti-colds and anti-influenza daily intake at first sign of infection:

| | | |
|---|---|---|
| 1,600 | mg | echinacea with goldenseal |
| 1,000 | mg | Cat's Claw (una de gato) |
| 1,000 | mg | pao d'arco |
| 1,500 | mg | elderberry dried berries |
| 1,000 | mg | olive leaf extract |
| 400 | mg | beta glucans |
| 450 | mg | cayenne pepper |

If a sore throat is present, the formula of the third embodiment is supplemented by 75 mg of zinc lozenges (zinc gluconite). The formula is taken daily with food. The formula can be taken in tablet form or as a tea, in which case the ingredients are placed in a container with eight ounces of water. To prepare the tea, the formula is brought to a boil, steeped five minutes, strained, and can be sweetened with honey.

The formulae of antiviral herbs can prevent the common cold and influenza by boosting the immune system. When taken as directed, the formulae can stop a cold or the flu or abate their duration and severity. The formulae of herbs protects against a wide variety of respiratory pathogens.

While there have been described specific embodiments of the invention, it will be clear that variations in the details of the embodiments specifically described may be made without departing from the true spirit and scope of the invention as defined in the appended claims.

I claim:

1. An herbal formulation for affecting colds or the flu in a human subject comprising beta glucans in combination with at least one of olive leaf extract, una de gato, pao d'arco, elderberry dried berries and cayenne pepper, the beta glucans and other herbs being of sufficient quantity to affect colds or the flu in humans.

2. The herbal formulation of claim 1 further including echinacea.

3. The herbal formulation of claim 1 further including echinacea and goldenseal.

4. The herbal formulation of claim 3 further including zinc gluconate.

5. The herbal formulation of claim 1 further including zinc gluconate.

6. A method of affecting a cold or the flu in a human subject comprising orally administering the herbal formulation of claim 1 to the subject.

7. An herbal formulation for affecting colds or the flu in a human subject comprising olive leaf extract in combination with at least one of beta glucans, una de gato, pao d'arco, elderberry dried berries and cayenne pepper, the olive leaf extract and other herbs being of sufficient quantity to affect colds or the flu in humans.

8. The herbal formulation of claim 7 further including echinacea.

9. The herbal formulation of claim 7 further including echinacea and goldenseal.

10. The herbal formulation of claim 9 further including zinc gluconate.

11. The herbal formulation of claim 7 further including zinc gluconate.

12. A method of affecting a cold or the flu in a human subject comprising orally administering the herbal formulation of claim 7 to the subject.

13. An herbal formulation for affecting colds or the flu in a human subject comprising beta glucans, olive leaf extract, una de gato, pao d'arco, elderberry dried berries, echinacea and cayenne pepper, the herbs being of sufficient quantity to affect colds or the flu in humans.

14. The herbal formulation of claim 13 further including goldenseal.

15. The herbal formulation of claim 14 further including zinc gluconate.

16. The herbal formulation of claim 13 further including zinc gluconate.

17. A method of affecting a cold or the flu in a human subject comprising orally administering the herbal formulation of claim 13 to the subject.

18. The method of claim 17 wherein the formulation includes goldenseal.

19. The method of claim 18 wherein the formulation includes zinc gluconate.

20. The method of claim 17 wherein the formulation includes zinc gluconate.
